(19) United States
(12) Patent Application Publication
(10) Pub. No.: US 2014/0346091 A1
(43) Pub. Date: Nov. 27, 2014

(54) PROCESSING MINED MATERIAL

(71) Applicants: Samuel Kingman, Rolleston on Dove (GB); Georgios Dimitrakis, Nottingham (GB); Christopher Dodds, West Bridgford (GB); Grant Ashley Wellwood, Midarie, Western Australia (AU)

(72) Inventors: Samuel Kingman, Rolleston on Dove (GB); Georgios Dimitrakis, Nottingham (GB); Christopher Dodds, West Bridgford (GB); Grant Ashley Wellwood, Midarie, Western Australia (AU)

(73) Assignee: TECHNOLOGICAL RESOURCES PTY. LIMITED, Brisbane, QLD (AU)

(21) Appl. No.: 14/235,893

(22) PCT Filed: Aug. 6, 2012
(86) PCT No.: PCT/AU2012/000923

§ 371 Date: Apr. 28, 2014

(30) Foreign Application Priority Data
Aug. 4, 2011 (AU) 2011903095

(51) Int. Cl. B07B 13/00; G21K 5/08; G01N 33/24; B07B 13/18; B07B 13/16

(52) U.S. Cl. CPC B07B 13/003; B07B 13/18; B07B 13/16; G01N 33/24; G21K 5/08
USPC 209/3.1; 250/453.11

(57) ABSTRACT

An apparatus for processing mined material that includes an applicator assembly (2) is disclosed. The applicator assembly includes a plurality of applicators (12) for exposing a moving bed of fragments of mined material to electromagnetic radiation as the bed of fragments moves through the applicator assembly. The applicators are arranged so that, in use, there is a high level of assurance that all of the fragments in the moving bed will receive at least a minimum exposure to electromagnetic radiation by the time the fragments reach an outlet end (8) of the applicator assembly.

### TECHNICAL FIELD

The present invention relates to a method and an apparatus for processing mined material.

The present invention also relates to an applicator for exposing fragments of mined material to electromagnetic radiation for use in the method and apparatus for processing mined material.

The term “mined” material is understood herein to include metalliferous material and non-metalliferous material. Iron-containing and copper-containing ores are examples of metalliferous material. Coal is an example of a non-metalliferous material. The term “mined” material is understood herein to include, but is not limited to, (a) run-of-mine material and (b) run-of-mine material that has been subjected to at least primary crushing or similar size reduction after the material has been mined and prior to being sorted. The mined material includes mined material that is in stockpiles.

The present invention relates particularly, although by no means exclusively, to a method and an apparatus for processing mined material to facilitate subsequent recovery of valuable material, such as valuable metals, from the mined material.

The present invention also relates to a method and an apparatus for recovering valuable material, such as valuable metals, from mined material that has been processed as described above.

The present invention relates particularly, although by no means exclusively, to a method and an apparatus for processing low grade mined material at high throughputs.

### BACKGROUND ART

The applicant is developing an automated sorting method and apparatus for mined material.

In general terms, the method of sorting mined material being developed by the applicant includes the following steps:

(a) exposing fragments of mined material to electromagnetic radiation,

(b) detecting and assessing fragments on the basis of composition (including grade) or texture or another characteristic of the fragments, and

(c) physically separating fragments based on the assessment in step (b).

Automated ore sorting technology known to the applicant is limited to low throughput systems. The general approach used in these low throughput sorting systems is to convey ore fragments through sorters on a horizontal belt. While horizontal conveyor belts are a proven and effective approach for fragments greater than 10 mm at throughputs up to around 200 t/h, the conveyor belts are unable to cater for the larger throughputs of 500-1000 t/h needed to realise the economies of scale required for many applications in the mining industry such as sorting low grade ore having particle sizes greater than 10 mm.

The applicant is also developing a method and an apparatus for forming microfractures in fragments of mined material by exposing the fragments to electromagnetic radiation. The microfractures in the fragments facilitate downstream processing of the fragments to recover valuable material, such as valuable metals, from the fragments. The downstream processing options include, by way of example, heap leaching, with the microfractures allowing leach liquor to penetrate the fragments and improve recovery of valuable metals. Another downstream processing option includes comminuting the fragments and forming smaller fragments, processing the smaller fragments in a flotation circuit and forming a concentrate and smelting the concentrate to recovery valuable metals. As is the case with ore sorting technology discussed above, the technology for forming microfractures in fragments of mined material known to the applicant is limited to low throughput systems.

An issue for the technology development paths of the applicant in the fields of sorting fragments and forming microfractures in fragments relates to processing mined material at high throughputs.

The above description is not to be understood as an admission of the common general knowledge in Australia or elsewhere.

### SUMMARY OF THE DISCLOSURE

In general terms, the present invention provides an apparatus for processing mined material, such as mined ore, that includes an applicator assembly including a plurality of applicators for exposing a moving bed of [symbol] as the bed of fragments moves through the applicator assembly, with the applicators being arranged so that, in use, there is a high level of assurance that all of the fragments in the moving bed will receive at least a minimum exposure to electromagnetic radiation by the time the fragments reach an outlet end of the applicator assembly.

The present invention is based on a realisation that providing an applicator assembly that includes multiple applicators arranged along a path of movement of a moving bed of fragments of mined material provides an opportunity to process high throughputs of mined material with a high level of assurance that all of the fragments in the moving bed will receive at least a minimum exposure to electromagnetic radiation that is required for downstream processing of the fragments. The applicant has realised that this high level of assurance may not be possible with a single applicator, particularly when operating at high throughputs of at least 200 tonnes per hour. In any given situation, the “minimum exposure” is a function of the downstream processing requirements for the fragments. In the context of ore sorting the term “minimum exposure” is understood herein to mean the minimum exposure to make downstream detection and assessment of the response of the fragments to electromagnetic radiation an accurate indication of the characteristic(s) of the fragments that are the basis for assessing the fragments. In the context of microfracturing fragments, the term “minimum exposure” is understood herein to mean the minimum exposure to form microfractures in the fragments that are required for downstream processing requirements, such as downstream crushing operations and heap leaching operations.

The term “fragment” is understood herein to mean any suitable size of mined material having regard to materials handling and processing capabilities of the apparatus used to carry out the method and the downstream processing requirements. In the context of ore sorting, relevant factors include issues associated with detecting sufficient information to make an accurate assessment of the mined material in the fragment. It is also noted that the term “fragment” as used herein may be understood by some persons skilled in the art to be better described as “particles”. The intention is to use both terms as synonyms.

The term “applicator” is understood herein to mean a chamber for receiving and retaining electromagnetic radiation within the chamber.

The term “bed” is understood herein to mean that adjacent fragments in the bed are in contact with each other.

The apparatus may include a separate source of electromagnetic radiation for each applicator.

The electromagnetic radiation may be pulsed or continuous electromagnetic radiation.

The applicator assembly may be adapted to operate with any suitable electromagnetic radiation. For example, the radiation may be any one or more of X-ray, microwave and radio frequency radiation.

In any given situation, the selection of the structure of the applicators and the electromagnetic radiation for the applicators, including the selection of [symbol] dependent on a number of factors including, but not limited to the mineralogy and composition of the mined material, the size distribution of the fragments, the transverse cross-sectional area of the bed of fragments, the rate of movement of the bed, the packing density in the bed, the purpose of the apparatus such as for sorting fragments or for micro-fracturing fragments' or for a combination of micro-fracturing and sorting fragments or for another purpose, the downstream processing route for the fragments (such as leaching, smelting, etc), and the characteristic(s) of the fragments to be assessed.

There may be situations in which it is desirable to expose fragments to radio frequency radiation initially in one or more than one applicator and to microwave radiation in one or more than one downstream applicator, and vice versa. In other situations, it may be desirable to operate each applicator with the same frequency of electromagnetic radiation. In other situations, it may be desirable to operate each applicator with different frequencies of electromagnetic radiation within the microwave radiation band.

In addition, in any given situation, the selection of the structure of the applicators and the electromagnetic radiation for the applicators, including the selection of the frequency and power density of the radiation for each of the applicators, is governed by the objective of providing a high level of assurance that all of the fragments in the moving bed will receive at least a minimum exposure to electromagnetic radiation that is required for downstream processing of the fragments.

Each applicator may be adapted to expose fragments moving through the assembly to electromagnetic radiation so that the combined effect of the operation of the applicators is that all of the fragments in the moving bed, across the transverse cross-sectional area of the moving bed at an outlet of the assembly, have received at least a predetermined minimum exposure to electromagnetic radiation.

This predetermined minimum exposure to electromagnetic radiation may be achieved by a range of different options for the applicators and the power densities generated by the applicators in the moving bed of fragments.

Each applicator may be adapted to operate across a whole or a part of transverse cross-sectional area of the moving bed.

The applicators may be positioned at spaced intervals along the length of the moving bed.

With this arrangement, the applicators may be at different orientations to the moving bed.

The applicators may be positioned at one position along the length of the moving bed, with each applicator being adapted to expose a part of the moving bed at that position to electromagnetic radiation.

For example, each applicator may be adapted to expose fragments to a minimum uniform power density across a transverse cross-sectional area of the bed so that the combined effect of the operation of the applicators is that all of the fragments in the moving bed, across the outlet of the assembly, have received at least a predetermined minimum exposure to electromagnetic radiation.

By way of further example, each applicator may be adapted to expose fragments to a range of power densities across a transverse cross-sectional area of the bed so that the combined effect of the operation of the applicators is that all of the fragments in the moving bed, across the transverse cross-sectional area of the moving bed at an outlet of the assembly, have received at least a predetermined minimum exposure to electromagnetic radiation.

By way of further example, each applicator may be adapted to expose fragments to a minimum uniform power density or to a range of power densities across a part of a transverse cross-sectional area of the bed as opposed to across the whole transverse cross-sectional area so that the combined effect of the operation of the applicators is that all of the fragments in the moving bed, across the transverse cross-sectional area of the moving bed at an outlet of the assembly, have received at least a predetermined minimum exposure to electromagnetic radiation.

The applicator assembly may include an applicator tube for containing the moving bed of fragments, with the applicator tube having an inlet and an outlet and being arranged to extend through each of the applicators in turn so that there is a series arrangement of applicators along the length of the tube.

In effect, such an arrangement can be described as an [symbol] series of microwave radiation applicator cavities, with the applicator tube being isolated from the cavities in a materials handling sense.

The applicator assembly may include an applicator tube for containing the moving bed of fragments, with the applicator tube having an inlet and an outlet and being arranged to extend through each of the applicators, with the applicators being arranged at the same position along the length of tube.

In use, mined material is processed in the applicator assembly on a bulk basis—as opposed to a fragment by fragment basis. More particularly, a feed mined material such as mined ore is supplied to the inlet of the applicator tube and moves as a bed of mined material, such as a packed bed in which the fragments are in contact with each other, through the applicator tube to the outlet of the tube. The fragments are exposed to electromagnetic radiation successively in each applicator as the fragments move from the inlet to the outlet of the applicator tube.

The applicator tube may be a wear resistant tube.

The applicator tube may be formed from a wear resistant material.

The applicator tube may include an inner lining of a wear resistant material.

The term “wear resistant” is understood herein in the context of the mined material being processed in the apparatus.

The applicator tube may be arranged horizontally.

The applicator tube may be arranged vertically or at an angle to the vertical and have an upper inlet and a lower outlet.

The angle may be in a range of up to 30° from the vertical.

The applicator tube may be at least 80 mm wide at the inlet.

The applicator tube may be at least 150 mm wide at the inlet.

The applicator tube may be at least 200 mm wide at the inlet.

The applicator tube may be at least 500 mm wide at the inlet.

The applicator tube may be at least 250 mm long.

The applicator tube may be at least 1 m long.

The applicator tube may be at least 2 m long.

The applicator tube may be no more than 5 m long.

The applicator tube may be any suitable transverse profile. By way of example, the tube may have a circular transverse cross-section.

The applicators may be at different orientations to the applicator tube.

The applicator assembly may be adapted to supply mined material to the applicator tube via gravity feed.

The applicator assembly may be adapted to supply mined material to the applicator tube via a forced feed.

The applicator assembly may include flow control assemblies upstream of the inlet and downstream of the outlet for controlling the flow of fragments into and from the applicator tube. The flow control assemblies may include rotary valves, such as a rotatable star wheel, and sliding gates.

The applicator assembly may also include chokes upstream of the inlet and downstream of the outlet for preventing electromagnetic radiation from escaping the applicator tube.

The applicator assembly may be adapted to operate on a continuous basis with mined material moving continuously through the applicator tube, for example in plug flow, and being exposed to electromagnetic radiation as it moves through the applicator.

In a situation where an applicator of the applicator assembly is adapted to operate with microwave radiation, a section of the applicator tube that is in the applicator may be transparent to electromagnetic radiation.

In a situation where an applicator of the applicator assembly is adapted to operate with radio frequency radiation, the applicator may include a first electrode within the applicator tube and a second electrode outside or forming at least a part of the applicator tube or both electrodes outside the tube.

The cross-sectional area of the applicator tube may be uniform along the length of the tube.

The cross-sectional area of the applicator tube may vary along the length of the tube. For example, the between the inlet and the outlet of the tube. By way of further example, the cross-sectional area of the applicator tube may be uniform for a first section of the tube extending from the inlet and then may increase continuously along the length of the remainder of the tube to the outlet of the tube.

According to the present invention there is provided an apparatus for sorting mined material, such as mined ore, that includes:

(a) an applicator assembly including a plurality of applicators for exposing a moving bed of fragments to electromagnetic radiation as the bed of fragments moves through the applicator assembly, with the applicators being arranged so that, in use, there is a high level of assurance that all of the fragments in the moving bed will receive at least a minimum exposure to electromagnetic radiation by the time the fragments reach an outlet end of the applicator assembly;

(b) a detection and assessment system for detecting and assessing one or more than one characteristic of the fragments, and

(c) a sorting means in the form of a separator for separating the fragments into multiple streams in response to the assessment of the detection and assessment system.

The applicator assembly may have the above-described features.

The apparatus's may include a fragment distribution assembly for distributing fragments from the applicator assembly so that the fragments move downwardly and assembly as individual, separate fragments that are not in contact with each other. The assembly may have an upper inlet and a lower outlet and a downwardly and outwardly extending distribution surface on which fragments are able to move from the upper inlet to the lower outlet and which allow fragments to be distributed into individual, separate fragments by the time the fragments reach the lower outlet. In use of this arrangement, fragments from the outlet of the applicator tube are supplied to the upper inlet of the fragment distribution assembly. The fragments move, for example by sliding and/or tumbling, down the distribution surface of the assembly. The fragments move downwardly and outwardly on the distribution surface from the upper inlet to the lower outlet of the distribution assembly. The distribution surface allows the fragments to disperse into a distributed state in which the fragments are not in contact with other fragments and move as individual, separate fragments and are discharged from the distribution assembly in this distributed state.

The distribution surface of the distribution assembly may be a conical surface or a segment of a conical surface that extends downwardly and outwardly.

The distribution surface may be an upper surface of a conical member or a segment of a conical member or an upper surface of a frusto-conical member or a segment of a frusto-conical member that are arranged to extend downwardly and outwardly.

The conical surface may define any suitable cone angle, i.e. any suitable angle to a horizontal axis.

The conical surface may define an angle of at least 30° to a horizontal axis.

The conical surface may define an angle of at least 45° to a horizontal axis.

The conical surface may define an angle of less than 75° to a horizontal axis.

The distribution surface of the distribution assembly may be an upper surface of an angled plate, such as an angled flat plate.

The distribution surface of the distribution assembly may be an upper surface of a pair of plates, such as a pair of flat plates or a pair of curved plates, that extend outwardly and downwardly away from each other.

The distribution assembly may include a chamber that is defined in part by the distribution surface.

The chamber may be a conical or a frusto-conical chamber.

The distribution assembly may be adapted to operate as a second electromagnetic radiation applicator assembly for exposing fragments to electromagnetic radiation as the fragments move down the distribution surface. In that event, the apparatus may include a source of electromagnetic radiation for the chamber. In use of such an arrangement the mined material is exposed to electromagnetic radiation in two applicator assemblies, namely this chamber, which is a form of an applicator, and the applicators in the upstream (in terms of the direction of movement of material) applicator assembly.

The same or different exposure conditions may be used in the two applicator assemblies, depending on the requirements in any given situation. For example, the electromagnetic radiation in the upstream applicator may be selected to cause microfracturing of the fragments to break down the fragments into smaller sizes and the electromagnetic radiation in the downstream distribution assembly may be selected to facilitate sorting of the fragments. In this arrangement, the operating conditions in the upstream applicator assembly may be selected, having regard to the characteristics of the mined material so that the fragments fracture to smaller fragments in the upstream applicator assembly and/or as the fragments move through the downstream distribution assembly and/or in downstream processing steps, such as conventional comminution steps. By way of further example, the electromagnetic radiation in one applicator assembly may be selected to allow detection and assessment of one characteristic and the other applicator may be selected to allow detection and assessment of another characteristic of the fragments.

The detection and assessment system may include a sensor for detecting the response, such as the thermal response, of each fragment to electromagnetic radiation.

The detection and assessment system may include a sensor for detecting other characteristics of the fragment. The sensor may include any one or more than one of the following sensors: (i) near-infrared spectroscopy (“NIR”) sensors (for composition), (ii) optical sensors (for size and texture), (iii) acoustic wave sensors (for internal structure for leach and grind dimensions), (iv) composition), and (v) magnetic property sensors (for mineralogy and texture); (vi) x-ray sensors for measurement of non-sulphidic mineral and gangue components, such as iron or shale. Each of these sensors is capable of providing information on the properties of the mined material in the fragments, for example as mentioned in the brackets following the names of the sensors.

The detection and assessment system may include a processor for analysing the data for each fragment, for example using an algorithm that takes into account the sensed data, and classifying the fragment for sorting and/or downstream processing of the fragment, such as flotation, heap leaching and smelting.

The assessment of the fragments may be on the basis of grade of a valuable metal in the fragments. The assessment of the fragments may be on the basis of another characteristic (which could also be described as a property), such as any one or more of hardness, texture, mineralogy, structural integrity, and porosity of the fragments. In general terms, the purpose of the assessment of the fragments is to facilitate sorting of the fragments and/or downstream processing of the fragments. Depending on the particular circumstances of a mine, particular combinations of properties may be more or less helpful in providing useful information for sorting of the fragments and/or downstream processing of the fragments.

The detection and assessment system may be adapted to generate control signals to selectively activate the separator in response to the fragment assessment.

The lower outlet of the distribution assembly may be adapted to discharge fragments as a downwardly-falling curtain of fragments. The curtain of material is a convenient form for high throughput analysis of fragments.

The separator for separating the fragments into multiple streams in response to the assessment of the detection and assessment system may be any suitable separator. By way of example, the separator may include a plurality of air jets that can be actuated selectively to displace fragments form a path of movement.

The apparatus may be adapted to sort mined material at any suitable throughput. The required throughput in any given situation is dependent on a range of factors including, but not limited to, operating requirements of upstream and downstream operations.

The apparatus may be adapted to sort at least 100 tonnes per hour of mined material.

The apparatus may be adapted to sort at least 250 tonnes per hour of mined material.

The apparatus may be adapted to sort at least 500 tonnes per hour of mined material.

The apparatus may be adapted to sort at least 1000 tonnes per hour of mined material.

The mined material may be any mined material that contains valuable material, such as valuable metals.

Examples of valuable materials are valuable metals in minerals such as minerals that comprise metal oxides or metal sulphides. Specific examples of valuable materials that contain metal oxides are iron ores and nickel laterite ores. Specific examples of valuable materials that contain metal sulphides are copper-containing ores. Other examples of valuable materials are salt and coal.

Particular, although not exclusive, areas of interest to the applicant are mined material in the form of (a) ores that include copper-containing minerals such as chalcopyrite, in sulphide forms and (b) iron ore.

The present invention is particularly, although not exclusively, applicable to sorting low grade mined material.

The term “low” grade is understood herein to mean that the economic value of the valuable material, such as a metal, in the mined material is only marginally greater than the costs to mine and recover and transport the valuable material to a customer.

In any given situation, the concentrations that are regarded as “low” grade will depend on the economic value of the valuable material and the mining and other costs to recover the valuable material from the mined material at a particular point in time. The concentration of the valuable material may be relatively high and still be regarded as “low” grade. This is the case with iron ores.

In the case of valuable material in the form of copper sulphide minerals, currently “low” grade ores are run-of-mine ores containing less than 1.0% by weight, typically less than 0.6 wt. %, copper in the ores. Sorting ores having such low concentrations of copper from barren fragments is a challenging task from a technical viewpoint, particularly in situations where there is a need to sort very large amounts of ore, typically at least 10,000 tonnes per hour, and where the barren fragments represent a smaller proportion of the ore than the ore that contains economically recoverable copper.

The term “barren” fragments, when used in the context of copper-containing ores, is understood herein to mean fragments with no copper or very small amounts of copper that cannot be recovered economically from the fragments.

The term “barren” fragments when used in a more general sense in the context of valuable materials is understood herein to mean fragments with no valuable material or amounts of valuable material that can not be recovered economically from the fragments.

According to the present invention there is provided an applicator assembly including a plurality of applicators for exposing a moving bed of fragments to electromagnetic radiation as the bed of fragments moves through the applicator assembly, with each applicator being adapted to expose fragments moving through the applicator assembly to a minimum power density (which equates to absorbed energy over a period of time) across a transverse cross-sectional area of the bed so that the combined effect of the operation of the applicators is that all of the fragments in the moving bed, across the transverse cross-sectional area of the moving bed at an outlet of the assembly, have received at least a minimum exposure to electromagnetic radiation.

Each applicator may be adapted to expose fragments moving through a section of the applicator assembly to a range of power densities across a transverse cross-sectional area of the bed so that the combined effect of [symbol] fragments in the moving bed, across the transverse cross-sectional area of the moving bed at an outlet of the assembly, have received at least a minimum exposure to electromagnetic radiation.

The applicator assembly may include an applicator tube for containing the moving bed of fragments, with the applicator tube having an inlet and an outlet and being arranged to extend through each of the applicators in turn so that there is a series arrangement of applicators along the length of the tube.

According to the present invention there is, provided a method of processing mined material, such as mined ore, including moving a bed of fragments of mined material through each of the applicators in the above-described applicator assembly and exposing the fragments to electromagnetic radiation as the fragments move through the applicator assembly so that there is a high level of assurance that all of the fragments in the moving bed will receive at least a minimum exposure to electromagnetic radiation by the time the fragments reach an outlet end of the applicator assembly.

The method may include operating the applicators so that all of the fragments in the moving bed receive at least a minimum exposure to electromagnetic radiation that is required for downstream processing of the fragments.

The method may include moving the fragments horizontally through the electromagnetic radiation applicator assembly.

The method may include moving the fragments downwardly through the electromagnetic radiation applicator assembly via a gravity feed.

The method may include moving the fragments downwardly through the electromagnetic radiation applicator assembly via a forced feed.

The method may include moving the fragments through the applicator at a speed of at least 0.5 m/s.

The method may include moving the fragments through the applicator at a speed of at least 0.6 m/s.

The method may include sorting mined material at a throughput of at least 100 tonnes per hour.

The method may include sorting mined material at a throughput of at least 250 tonnes per hour.

The method may include sorting mined material at a throughput of at least 500 tonnes per hour.

The method may include sorting mined material at a throughput of at least 1000 tonnes per hour.

According to the present invention there is provided a method of sorting mined material, such as mined ore, including the steps of:

(a) moving a bed of fragments of mined material through each of the applicators in the above-described electromagnetic radiation applicator assembly and exposing the fragments to electromagnetic radiation as the fragments move through the applicator assembly so that there is a high level of assurance that all of the fragments in the moving bed will receive at least a minimum exposure to electromagnetic radiation by the time the fragments reach an outlet end of the applicator assembly,

(b) detecting one or more than one characteristic of the fragments,

(c) assessing the characteristic(s) of the fragments, and

(d) sorting the fragments into multiple streams in response to the assessment of the characteristic(s) of the fragments.

The method may include supplying the fragments that have been exposed to electromagnetic radiation to a distribution assembly and allowing the fragments to move downwardly and outwardly over a distribution surface of the assembly from an upper inlet to a lower outlet so that the fragments are distributed into individual, separate fragments and are discharged from the assembly as individual, separate fragments.

The method may include exposing the fragments to electromagnetic radiation as the fragments move downwardly and outwardly over the distribution surface of the distribution assembly.

Method step (a) may be as described above in relation to the more general method of processing mined material.

Detection step (b) may include detecting the response, such as the thermal response, of each fragment to exposure to electromagnetic radiation.

Assessment step (c) may include analysing the response of each fragment to identify valuable material in the fragment.

Detection step (b) is not confined to sensing the response of fragments of the mined material to electromagnetic radiation and extends to sensing additional characteristics of the fragments. For example, step (b) may also extend to the use of any one or more than one of the following sensors: (i) near-infrared spectroscopy (“NIR”) sensors (for composition), (ii) optical sensors (for size and texture), (iii) acoustic wave sensors (for internal structure for leach and grind dimensions), (iv) laser induced spectroscopy (“LIBS”) sensors (for composition), and (v) magnetic property sensors (for mineralogy and texture); (vi) x-ray sensors for measurement of non-sulphidic mineral and gangue components, such as iron or shale. Each of these sensors is capable of providing information on the properties of the mined material in the fragments, for example as mentioned in the brackets following the names of the sensors.

The method may include a downstream processing step of comminuting the sorted material as a pre-treatment step for a downstream option for recovering the valuable mineral from the mined material.

The method may include a downstream processing step of blending the sorted material as a pre-treatment step for a downstream option for recovering the valuable mineral from the mined material.

The method may include using the sensed data for each processing options, such as flotation and comminution, and as feed-back information to upstream mining and processing options.

The upstream mining and processing options may include drill and blast operations, the location of mining operations, and crushing operations.

According to the present invention there is also provided a method for recovering valuable material, such as a valuable metal, from mined material, such as mined ore, that includes processing mined material according to the method described above and thereafter further processing the fragments containing valuable material and recovering valuable material.

The further processing options for the processed fragments may be any suitable options, such as smelting and leaching options.

### BRIEF DESCRIPTION OF THE DRAWINGS

The present invention is described further by way of example with reference to the accompanying drawings of which:

FIG. 1 illustrates diagrammatically a vertical cross-section of key components of one embodiment of a sorting apparatus in accordance with the present invention, which includes one embodiment of an electromagnetic radiation applicator assembly in accordance with the present invention;

FIG. 2(a) is a perspective view of the embodiment of the applicator assembly shown in FIG. 1,

FIG. 2(b) is a map of the power density distribution through a vertical cross-section of the applicator assembly shown in FIGS. 1 and 2(a), across the width and along the length of the assembly; and

FIG. 3 is a perspective view of another embodiment of an apparatus for processing mined material in accordance with the present invention, with this embodiment being concerned with microfracturing fragments of mined material rather than sorting mined material as is the case with the FIG. 1 embodiment.

### DESCRIPTION OF EMBODIMENTS

The embodiments are described in the context of the use of microwave radiation as the electromagnetic radiation. However, it is noted that the invention is not confined to the use of microwave radiation and extends to the use of other types of electromagnetic radiation, such as radio frequency radiation and x-ray radiation. In addition, it is noted that the present invention extends to operating with combinations of frequencies across the electromagnetic radiation spectrum and is not confined to operating with what are described as the microwave, radiation and radio frequency radiation and x-ray radiation bands.

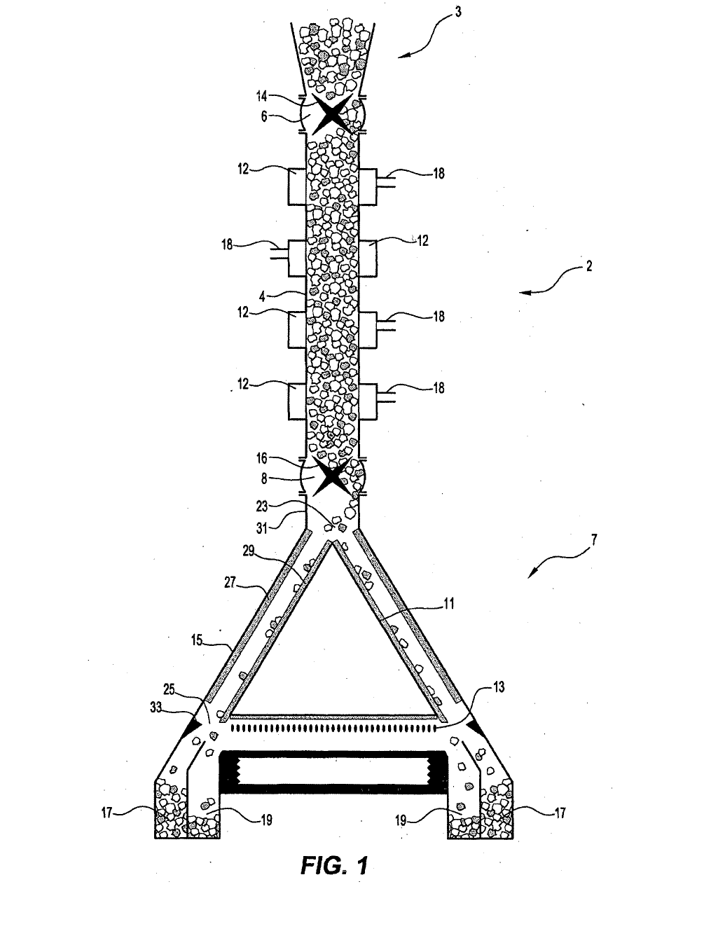

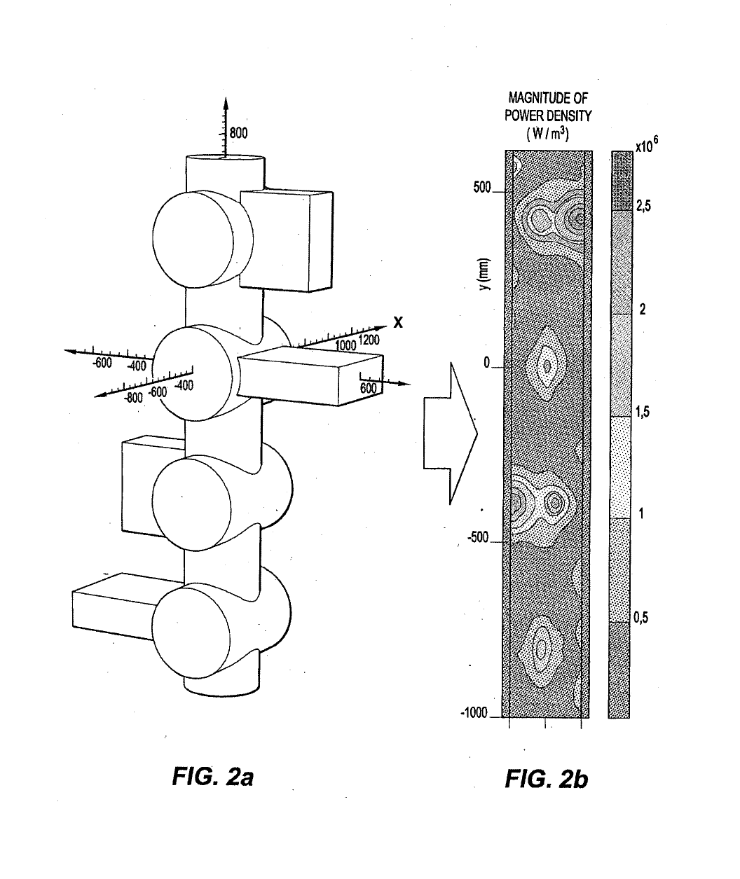

The embodiment of the method of processing mined masterial shown in FIGS. 1 and 2 is described as a method of sorting mined material. More particularly, the embodiment is described in the context of a method and an apparatus for recovering a valuable metal in the form of copper from a low grade copper-containing ore in which the copper is present in copper-containing minerals such as gangue. The objective of the method in this embodiment is to identify fragments of mined material containing amounts of copper-containing minerals above a certain grade and to sort these fragments from the other fragments and to process the copper-containing fragments as required to recover copper from the fragments.

It is noted that, whilst the following description does not focus on the downstream processing options, these options are any suitable options ranging from smelting to leaching or comminution and flotation of the fragments.

It is also noted that whilst the following description focuses on sorting mined material, the invention also extends to other processing options, such as microfracturing fragments of mined material.

It is also noted that the present invention is not confined to copper-containing ores and to copper as the valuable material to be recovered. In general terms, the present invention provides a method of sorting any minerals which exhibit different heating responses when exposed to electromagnetic radiation.

With reference to FIG. 1, a feed material in the form of fragments of copper-containing ore that have been crushed by a primary crusher (not shown) to a fragment size of 10-25 cm is supplied under gravity feed via a vertical transfer hopper 3 (or other suitable transfer means, such as a conveyor belt supplying material to a feed hopper) to a microwave radiation applicator assembly generally identified by the numeral 2.

The applicator assembly 2 includes a vertical cylindrical chute or tube 4. The ore is exposed to microwave radiation on a bulk basis as the fragments move downwardly in a bed, preferably a packed bed in which the fragments are in contact moving in plug flow, through the tube 4 from an upper inlet 6 to a lower outlet 8 of the tube 4. The tube 4 is formed from a wear resistant material and includes a lining of a dielectric material. By way of example, the tube 4 is formed from a wear resistant ceramic material. As is described in more detail below, sections of the tube are transparent to microwave radiation and other sections of the tube are not transparent to microwave radiation.

As can best be seen in FIG. 2, the applicator assembly 2 also includes a plurality of microwave radiation applicators 12, with the applicators 12 and the tube 4 being arranged so that the tube 4 extends through each of the applicators 12, whereby the applicators 12 are spaced apart and in a series arrangement along the path of movement of fragments through the applicator assembly 2. The arrangement is such that the sections of the tube 4 that are transparent to microwave radiation are enclosed by the applicators 12 and the sections of the tube 4 that are between the applicators 12 are not transparent to microwave radiation. Each applicator 12 includes a waveguide 18 for transferring microwave radiation to the applicator 12. Each applicator 12 may include any suitable number of waveguides.

In FIG. 2(a) the waveguides 18 extend perpendicular to the longitudinal axis of the tube 4. The waveguides 18 could be positioned at any suitable angle to the tube axis in order to optimise performance of the apparatus. For example, the waveguides 18 could be positioned at suitable dielectric properties of the lining material to minimise reflection of microwave radiation from the lining material. In addition, the thickness of the dielectric lining may be selected to facilitate better power matching to the material.

In the arrangement shown in FIG. 2, each applicator 12 extends around the whole circumference of the section of the length of the tube 4 at which the applicator is positioned and thereby defines a chamber around this section of the tube. It is noted that the present invention is not limited to this arrangement and one or more than one of these applicators 12 could be formed to enclose a segment of the circumference of section of the length of the tube 4 and thereby define a chamber in relation to this segment of the section of the tube. There could also be arrangements in which there is a plurality of separate applicators 12 at each of a number of positions along the length of the tube 4, with each of these applicators 12 being formed to enclose a segment of the circumference of a section of the length of the tube 4 and thereby define a chamber in relation to this segment of the section of the tube.

As can best be seen in FIG. 2(a), the applicators 12 have different shapes and different orientations of the waveguides 18 with respect to the circumference of the tube 4. The present invention is not confined to these particular shapes and waveguide orientations of applicators 12 or to this order of shapes of applicators 12. The shapes and the order of the applicators 12 and the waveguide orientations and the frequency and other operating parameters for the microwaves for the [symbol] is a function of a range of factors including, but not limited to, the mineralogy and composition of the mined material, the size distribution of the fragments, the transverse cross-sectional area of the bed of fragments, the rate of movement of the bed, the purpose of the apparatus such as for sorting fragments or for microfracturing fragments or for a combination of microfracturing and sorting fragments or for another purpose, the downstream processing route for the fragments (such as leaching, smelting, etc), and the characteristic(s) of the fragments to be assessed.

In the described embodiment, the selection of shapes and the arrangement of the applicators 12 and the wave guide orientations and the frequency and other microwave radiation operating parameters and the size and other parameters of the applicator tube 4 are governed by the objective of processing high throughputs of mined material so that all of the fragments in the bed moving through the applicator assembly 2 receive at least a minimum exposure to electromagnetic radiation that is required for reliable downstream assessment of selected characteristics of the fragments and sorting of the fragments based on the assessment.

FIG. 2(b) is a map of the power density distribution through a vertical cross-section of the applicator assembly 2 shown in FIGS. 1 and 2(a), across the width and along the length of the assembly under a specific set of test conditions. The map illustrates the effectiveness of the embodiment. The map is shaded to indicate the power densities through the tube 8 at this cross-section—see the scale on the right side of Figure fragments. It is evident from the map that different sections of the applicator tube 4 receive considerably higher power densities of microwave radiation than other sections of the tube 4. As a consequence, fragments moving through these “hotter” sections will receive significantly higher heating loads than in other sections of the tube 4. It is also evident from the map that the distribution of “hotter” sections across the width and along the length of the tube 4 is such that every fragment moving through this vertical cross-section of the tube 4 will be exposed to high power density microwave radiation by the time that the fragments reach the outlet end 8 of the tube 4. As a consequence, all of the fragments in the moving bed receive at least a minimum exposure to electromagnetic radiation required for downstream processing of the fragments. In this embodiment the downstream processing involves making downstream detection and assessment of the response of the fragments to microwave radiation an accurate indication of the characteristic(s) of the fragments that are the basis for assessing the fragments.

It is noted that the map shown in FIG. 2(b) is representative of the power density distribution in the tube 4.

It is also noted that the map is illustrative of one of a number of possible arrangements of applicators 12 and operating conditions that achieve the objective of processing high throughputs of mined material so that all of the fragments in a bed of material moving through the applicator assembly 2 receive at least a minimum exposure to electromagnetic radiation required for downstream processing, in this instance for sorting material. More possible arrangements of applicators 12 and operating conditions that could achieve this objective.

With further reference to FIG. 1, chokes 14, 16 for preventing microwave radiation escaping from the tube 4 are positioned upstream of the inlet 6 and downstream of the outlet 8 of the tube 4. The chokes 14, 16 are in the form of rotary valves in the form of rotatable star wheels in this instance (as shown diagrammatically in the Figure) that also control supply and discharge of ore into and from the tube 4.

The outlet 8 of the tube 4 is aligned vertically with an inlet of a fragment distribution assembly. The distribution assembly is generally identified by the numeral 7. The outlet 8 supplies fragments that have been exposed to microwave radiation in the tube 4 directly to the distribution assembly 7.

The distribution assembly 7 includes a distribution surface 11 for the fragments. The fragments move downwardly and outwardly over the distribution surface 11, typically in a sliding and/or a tumbling motion, from an upper central inlet 23 of the distribution assembly 7 to a lower annular outlet 25 of the assembly 7. The distribution surface 11 allows the fragments to disperse from the packed bed state in which the fragments are in contact with each other in the tube 4 to a distributed state in which the fragments are not in contact with other fragments and move as individual, separate fragments and are discharged from the outlet 25 as individual, separate fragments.

The distribution assembly 7 comprises an inner wall [symbol] surface 11. The conical surface is an upper surface of a conical-shaped member.

The distribution surface 11 is shrouded by an outer wall having a second concentric outer conical surface 15. The distribution assembly 7 also includes chokes 31, 33 in the upper inlet 23 and the lower outlet 25 of the assembly 7. As a consequence, if required from an operational viewpoint, the assembly 7 may function as a second applicator for further exposing the fragments to electromagnetic radiation. The electromagnetic radiation may be microwave radiation or any other suitable type of radiation. Depending on the circumstances, the apparatus may include another source of electromagnetic radiation in addition to that forming part of the applicator assembly 2. In this context, this configuration of the apparatus has a particular advantage in the case of electromagnetic radiation in the radio frequency band. When operating with radio frequency radiation, the distribution surface 11 and the outer conical surface 15 are electrically isolated and configured to form parallel electrodes of a radio frequency applicator. These electrodes are identified by the numerals 27, 29 in FIG. 1.

The fragments are detected and assessed by a detection and assessment system as they move through the distribution assembly 7.

More specifically, while passing through the distribution assembly 7, radiation, more particularly heat radiation, from the fragments as a consequence of (a) exposure to microwave energy at the assembly 2 and optionally in the distribution assembly 7 and (b) the characteristics (such as composition and texture) of the fragments is detected by thermal imagers in the form of high resolution, high speed infrared imagers (not shown) which capture thermal images of the fragments. While one thermal imager is sufficient, two or more thermal imagers may be used for full coverage of the fragment surface. It is noted that the present invention is not limited to the use of such high resolution, high speed infrared imagers. It is also noted that the present invention is not limited to detecting the thermal response of fragments to microwave energy and extends to detecting other types of response.

From the number of detected hot spots (pixels), temperature, pattern of their distribution and their cumulative area, relative to the size of the fragments, an estimation of the grade of the fragments can be made. This estimation may be supported and/or more mineral content may be quantified by comparison of the data with previously established relationships between microwave induced thermal properties of specifically graded and sized fragments.

In addition, one or more optical sensors, for example in the form of visible light cameras (not shown) capture visible light images of the fragments to allow determination of fragment size.

The present invention also extends to the use of other sensors for detecting other characteristics of the fragments, such as texture.

Images collected by the thermal imagers and the visible light cameras (and information from other sensors that may be used) are processed in the detection and by the word “Control System”) equipped with image processing and other relevant software. The software is designed to process the sensed data to assess the fragments for sorting and/or downstream processing options. In any given situation, the software may be designed to weight different data depending on the relative importance of the properties associated with the data.

The detection and assessment system generates control signals to selectively activate a sorting means in response to the fragment assessment.

More specifically, the fragments free-fall from the outlet 25 of the distribution assembly 7 and are separated into annular collection bins 17, 19 by a sorting means that comprises compressed air jets (or other suitable fluid jets, such as water jets, or any suitable mechanical devices, such as mechanical flippers) that selectively deflect the fragments as the fragments move in a free-fall trajectory from the outlet 25 of the distribution assembly 7. The air jet nozzles are identified by the numeral 13. The air jets selectively deflect the fragments into two circular curtains of fragments that free-fall into the collection bins 17, 19. The thermal analysis identifies the position of each of the fragments and the air jets are activated a pre-set time after a fragment is analysed as a fragment to be deflected.

The positions of the thermal imagers and the other sensors and the computer and the air jets may be selected as required. In this connection, it is acknowledged that the figure is not intended to be other than a general diagram of one embodiment of the invention.

The microwave radiation may be either in the form of continuous or pulsed radiation. The microwave radiation may be applied at an electric field below that which is required to induce micro-fractures in the fragments. In any event, the microwave frequency and microwave intensity and the fragment exposure time and the other operating parameters of the assembly 2 are selected having regard to the information that is required. The required information is information that is required to assess the particular mined material for sorting and/or downstream processing of the fragments. In any given situation, there will be particular combinations of characteristics, such as grade, mineralogy, hardness, texture, structural integrity, and porosity, that will provide the necessary information to make an informed decision about the sorting and/or downstream processing of the fragments, for example, the sorting criteria to suit a particular downstream processing option.

As noted above, there may be a range of other sensors (not shown) other than thermal imagers and visible light cameras mentioned above positioned within and/or downstream of the assembly 2 and the distribution assembly 7 to detect other characteristics of the fragments depending on the required information to classify the fragments for sorting and/or downstream processing options.

In one mode of operation the thermal analysis is based on distinguishing between fragments that are above and below a threshold temperature. The fragments can then be categorised as “hotter” and “colder” fragments. The temperature of a fragment is related to the amount of [symbol] have a given size range and are heated under given conditions will have a temperature increase to a temperature above a threshold temperature “x” degrees if the fragments contain at least “y” wt. % copper. The threshold temperature can be selected initially based on economic factors and adjusted as those factors change. Barren fragments will generally not be heated on exposure to radio frequency radiation to temperatures above the threshold temperature.

In the present instance, the primary classification criteria is the grade of the copper in the fragment, with fragments above a threshold grade being separated into collection bin 19 and fragments below the threshold grade being separated into the collection bin 17. The valuable fragments in bin 19 are then processed to recover copper from the fragments. For example, the valuable fragments in the bin 19 are transferred for downstream processing including milling and flotation to form a concentrate and then processing the concentrate to recover copper.

The fragments in collection bin 17 may become a by-product waste stream and are disposed of in a suitable manner. This may not always be the case. The fragments have lower concentrations of copper minerals and may be sufficiently valuable for recovery. In that event the colder fragments may be transferred to a suitable recovery process, such as leaching.

Advantages of the present invention include the following advantages.
Processing ore fragments in bulk form in the applicator assembly 2 has been found to dramatically a horizontal belt arrangement with a mono-layer of mined material.
The use of an applicator assembly 2 that includes multiple applicators 12 arranged in series along a path of movement of a moving bed of fragments of mined material provides, an opportunity to process high throughputs of mined material with a high level of assurance that all of the fragments in the moving bed will receive at least a minimum exposure to electromagnetic radiation that is required for reliable downstream processing, such as a minimum exposure required for reliable assessment of selected characteristics of the fragments and sorting of the fragments based on the assessment.
The use of multiple applicators 12 simplifies the design of the apparatus. There is a significantly greater range of design options to meet the different processing challenges presented by different types of mined material, particularly when high throughput operation is required. Selecting combinations of smaller applicators is likely to be a far more cost-effective and reliable option than designing significantly larger single applicators in many instances.

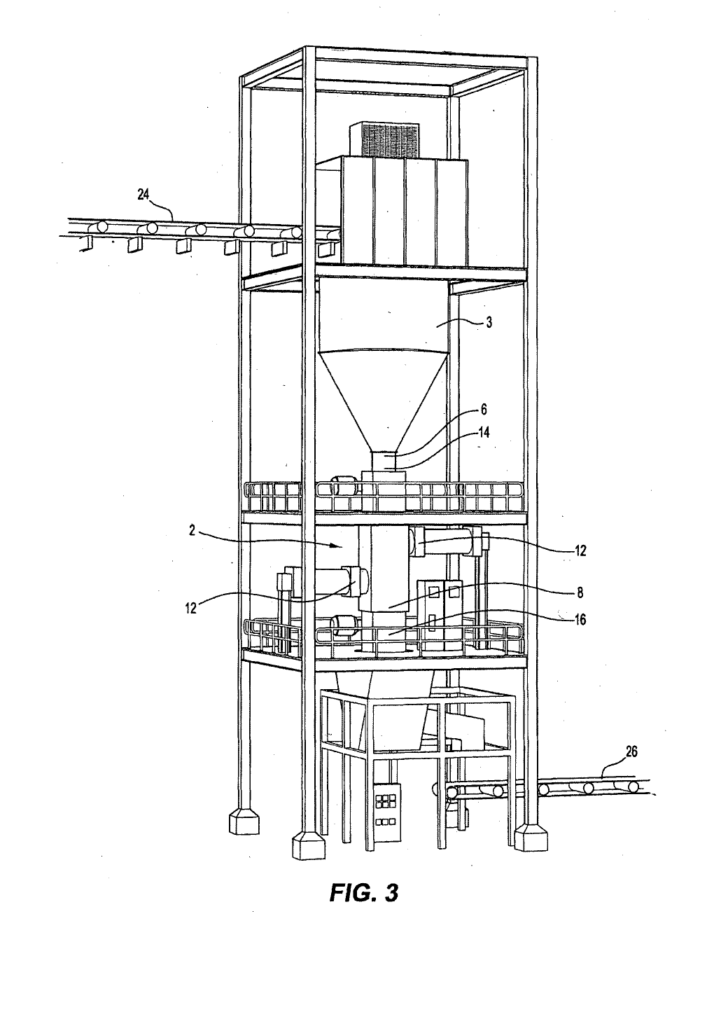

FIG. 3 is a perspective view of another, although not the only other possible, embodiment of an apparatus for processing mined material in accordance with the present invention, with this embodiment being concerned with microfracturing fragments of mined material to facilitate downstream processing of the fragments. The downstream processing may include comminuting the fragments and forming smaller fragments, processing the smaller fragments in a flotation circuit and forming a concentrate and smelting the concentrate to recovery valuable metals. Another downstream processing option includes heap leaching, with the microfractures allowing leach liquor to penetrate the fragments and improve recovery of valuable metals.

With reference to FIG. 3, a feed material in the form of fragments of copper-containing ore that have been crushed by a primary crusher (not shown) to a fragment size of 10-25 cm is supplied via a horizontal conveyor assembly 24 to a vertical transfer hopper 3 and then downwardly under gravity feed to a microwave radiation applicator assembly generally identified by the numeral 2. The applicator assembly 2 includes a vertical cylindrical tube 4 and two microwave radiation applicators 12 positioned along the length of the assembly 2. The ore is exposed to microwave radiation on a bulk basis as the fragments move downwardly in a bed, preferably a packed bed, through the tube 4 from an upper inlet 6 to a lower outlet 8 of the tube 4. Chokes 14, 16 for preventing microwave radiation escaping from the tube 4 are positioned upstream of the inlet 6 and downstream of the outlet 8 of the tube 4. The chokes 14, 16 are in the form of rotary valves that also control supply and discharge of ore into and from the tube 4. The ore discharged from the lower outlet 8 of the tube 4 is transferred onto a conveyor 26 or other suitable transfer option for downstream processing.

As is the case with the embodiment described in relation to FIGS. 1 and 2, the selection of shapes and [symbol] orientations and the frequency and other microwave radiation operating parameters and the size and other parameters of the applicator tube 4 are governed by the objective of processing high throughputs of mined material with a high level of assurance that all of the fragments in the bed moving through the applicator assembly 2 receive at least a minimum exposure to electromagnetic radiation that is required for downstream processing of the fragments.

Many modifications may be made to the embodiment of the present invention described above without departing from the spirit and scope of the present invention.

By way of example, the present invention is not limited to the use of an applicator tube 4 to contain the moving bed of fragments.

In addition, the present invention is not limited to the use of a vertical applicator tube 4.

In addition, the present invention is not limited to a fragment by fragment detection and assessment and sorting of mined material and extends to bulk assessment and detection and sorting of mined material.

In addition, in situations where there is fragment by fragment detection and assessment and sorting of mined material, the present invention is not limited to the particular fragment distribution assembly 7 shown in FIG. 1.

1. An apparatus for processing mined material that includes an applicator assembly including an applicator tube for containing a moving bed of fragments, the applicator tube extending vertically or at an angle to the vertical and having an upper inlet and a lower outlet and chokes upstream of the inlet and downstream of the outlet for preventing electromagnetic radiation from escaping the applicator tube, a plurality of applicators for exposing the moving bed of fragments of mined material to electromagnetic radiation as the bed of fragments moves through the applicator tube, and a separate source of electromagnetic radiation for each applicator, wherein each applicator is adapted to expose fragments moving through the assembly to electromagnetic radiation so that the combined effect of the operation of the applicators in use of the applicator assembly is that all of the fragments in the moving bed, across the transverse cross-sectional area of the moving bed, receive at least a predetermined minimum exposure to electromagnetic radiation by the time the fragments reach the outlet end of the applicator tube.

2. (canceled)

3. The apparatus defined in claim 1 wherein the applicator assembly is adapted to operate with electromagnetic radiation selected from any one or more of X-ray, microwave and radio frequency radiation.

4. (canceled)

5. The apparatus defined in claim 1 wherein each applicator is adapted to operate across a whole or a part of transverse cross-sectional area of the moving bed.

6. The apparatus defined in claim 1 wherein the applicators are positioned at spaced intervals along the length of the moving bed.

7. The apparatus defined in claim 1 wherein the applicators are positioned at the same position along the length of the moving bed, with each applicator being adapted to expose a part of the moving bed at that position to electromagnetic radiation.

8-9. (canceled)

10. The apparatus defined in claim 1 wherein the applicator tube is a wear resistant tube.

11. (canceled)

12. The apparatus defined in claim 1 wherein the applicator tube is at least 80 mm wide at the inlet.

13. The apparatus defined in claim 1 wherein the applicator tube is at least 1 m long.

14. The apparatus defined in claim 1 wherein the applicators are at different orientations to the applicator tube.

15. The apparatus defined in claim 1 wherein the applicator assembly is adapted to supply mined material to the applicator tube via gravity feed.

16. The apparatus defined in claim 1 wherein the applicator assembly is adapted to supply mined material to the applicator tube via a forced feed.

17. The apparatus defined in claim 1 wherein the applicator tube includes flow control assemblies upstream of the inlet and downstream of the outlet for controlling the flow of fragments into and from the applicator tube.

18. (canceled)

19. An apparatus for sorting mined material that includes:
(a) an applicator assembly including an applicator tube for containing a moving bed of fragments, the applicator tube extending vertically or at an angle to the vertical and having an upper inlet and a lower outlet and chokes upstream of the inlet and downstream of the outlet for preventing electromagnetic radiation from escaping the applicator tube, a plurality of applicators for exposing the moving bed of fragments to electromagnetic radiation as the bed of fragments moves through the applicator tube, and a separate source of electromagnetic radiation for each applicator, wherein each applicator is adapted to expose fragments moving through the assembly to electromagnetic radiation so that the combined effect of the operation of the applicators in use of the applicator assembly is that all of the fragments in the moving bed, across the transverse cross-sectional area of the moving bed, receive at least a predetermined minimum exposure to electromagnetic radiation by the time the fragments reach the outlet end of the applicator tube,
(b) a detection and assessment system for detecting and assessing one or more than one characteristic of the fragments, and
(c) a sorting means in the form of a separator for separating the fragments into multiple streams in response to the assessment of the detection and assessment system.

20. The apparatus defined in claim 19 includes a fragment distribution assembly for distributing fragments from the applicator assembly so that the fragments move downwardly and outwardly and are discharged from the distribution assembly as individual, separate fragments that are not in contact with each other.

21. The apparatus defined in claim 20 wherein the distribution assembly has an upper inlet and a lower outlet and a downwardly and outwardly extending distribution surface on which fragments are able to move from the upper inlet to the lower outlet and which allow fragments to be distributed into individual, separate fragments by the time the fragments reach the lower outlet.

22. The apparatus defined in claim 19 wherein the detection and assessment system includes a sensor for detecting the response, such as the thermal response, of each fragment to electromagnetic radiation.

23. The apparatus defined in claim 19 wherein the detection and assessment system includes a processor for analysing the data for each fragment and classifying the fragment for sorting and/or downstream processing of the fragment, such as heap leaching and smelting.

24. An applicator assembly including an applicator tube for containing a moving bed of fragments, the applicator tube extending vertically or at an angle to the vertical and having an upper inlet and a lower outlet and chokes upstream of the inlet and downstream of the outlet for preventing electromagnetic radiation from escaping the applicator tube, a plurality of applicators for exposing a moving bed of fragments to electromagnetic radiation as the bed of fragments moves through the applicator tube, and a separate source of electromagnetic radiation for each applicator, with each applicator being adapted to expose fragments moving through the applicator assembly to a minimum power density across a transverse cross-sectional area of the bed so that the combined effect of the operation of the applicators in use of the applicator assembly is that all of the fragments in the moving bed across the transverse cross-sectional area of the moving bed receive at least a minimum exposure to electromagnetic radiation by the time the fragments reach the outlet end of the applicator tube.

25. The applicator assembly defined in claim 24 includes an applicator tube for containing the moving bed of fragments, with the applicator tube having an inlet and an outlet and being arranged to extend through each of the applicators in turn so that there is a series arrangement of applicators along the length of the tube.

26. A method of processing mined material including moving a bed of fragments of mined material through each of the applicators in the applicator assembly defined in claim 24 and exposing the fragments to electromagnetic radiation as the fragments move through the applicator assembly so that there is a high level of assurance that all of the fragments in the moving bed will receive at least a minimum exposure to electromagnetic radiation by the time the fragments reach an outlet end of the applicator tube.

27. The method defined in claim 26 includes operating the applicators so that the combined effect of the operation of the applicators is that all of the fragments in the moving bed receive at least a minimum exposure to electromagnetic radiation that is required for downstream processing of the fragments.

28. The method defined in claim 26 includes moving the fragments downwardly through the electromagnetic radiation applicator assembly via a gravity feed or via a forced feed.

29. The method defined in claim 26 includes moving the fragments through the applicator at a speed of at least 0.5 m/s.

30. The method defined in claim 26 includes sorting mined material at a throughput of at least 250 tonnes per hour.

31. A method of sorting mined material including the steps of:
(a) moving a bed of fragments of mined material through each of the applicators in the electromagnetic radiation applicator assembly defined in claim 24 and exposing the fragments to electromagnetic radiation as the fragments move through the applicator assembly so that there is a high level of assurance that all of the fragments in the moving bed will receive at least a minimum exposure to electromagnetic radiation by the time the fragments reach the outlet end of the applicator tube,
(b) detecting one or more than one characteristic of the fragments,
(c) assessing the characteristic(s) of the fragments, and
(d) sorting the fragments into multiple streams in response to the assessment of the characteristic(s) of the fragments.

32. A method for recovering valuable material from mined material that includes processing mined material according to the method defined in claim 26 and thereafter further processing the fragments containing valuable material and recovering valuable material.
